(19) United States
(12) Patent Application Publication
(10) Pub. No.: US 2004/0025873 A1
(43) Pub. Date: Feb. 12, 2004

(54) 3-In-1 in- line nebulizer, mediport dispenser and suction chamber for bag valve masks, endotracheal tubes and tracheotomy tubes

(76) Inventor: Edward Padgett, Washington, DC (US)

Correspondence Address: John Dodds, Dodds & Associates, 1707 N Street, NW, Washington, DC, 20036, US

(21) Appl. No.: 10/404,948

(22) Filed: Apr. 1, 2003

Related U.S. Application Data
(60) Provisional application No. 60/371,242, filed on Apr. 9, 2002.

(51) Int. Cl. A61M 16/00; A61M 16/10

(52) U.S. Cl. 128/203.12; 128/203.11; 128/206.21

(57) ABSTRACT

This invention constitutes a 3-in-1 in-line nebulizer, mediport dispenser, and suction chamber for bag valve masks, endotracheal tubes and tracheotomy tubes attachment with universal adaptability for airways for adults and children. The invention's nebulizer chamber (4) permits administering in-line respiratory medications. The invention's medication administration port (3) permits administering cardiac and narcotic antagonist medications down the endotracheal tube or tracheotomy tube to the patient without the need of interrupting ventilation or breathing assistance. The invention's suction port (4) facilitates the removal of emesis from the oral airway of the patient, endotracheal tube or tracheotomy tube without jeopardizing ventilation, breathing assistance to the patient or compromising endotracheal tube or tracheotomy tube placement.

### CROSS REFERENCE TO RELATED APPLICATIONS

This application claims the benefit of U.S. Provisional Patent Application Serial No. 60/371,242 filed on Apr. 9, 2002.

### STATEMENT REGARDING FEDERALLY SPONSORED RESEARCH OR DEVELOPMENT

Not applicable

### REFERENCE OF TO A “SEQUENCE LISTING”, A TABLE OR COMPUTER PROGRAM LISTING MATERIAL SUBMITTED ON A COMPACT

Not applicable

### BACKGROUND OF THE INVENTION

(1) Field of Invention

This invention relates to the field of emergency medical first aid, ambulatory and emergency room devices. This invention relates most particularly to medical patient nebulizers, mediport dispensers and suction medical inhalation devices used by medical first aid personnel, in ambulances and hospital and clinic emergency rooms, for patients' cardiopulmonary resuscitation and respiratory emergencies.

Most devices present in the prior art are medical respiratory masks, inhalation devices or nebulizers. Nevertheless, no device in the prior art comprises all the characteristics and uses of the invention subject of this application. Moreover, the 3-in-1 in-line nebulizer, mediport dispenser and suction chamber for bag valve masks, endotracheal tubes and tracheotomy tubes has universal adaptability and therefore may be used as airway devices for adults and children with probable or actual breathing difficulties.

The 3-in-1 in-line nebulizer, mediport dispenser and suction chamber for bag valve masks, endotracheal tubes and tracheotomy tubes has been designed primarily to be used as:

(i) an adjunct to bag valve masks for dispensing cardiac medications and narcotic antagonists through an endotracheal tube;

(ii) as a device to dispense respiratory medications such as bronchodilators through a nebulizer dispenser or aerosolizer; and

(iii) as a device including a retractable suction chamber that will facilitate the foreign body or emesis removal from the oral airway, endotracheal tube or tracheotomy tube without jeopardizing ventilation assistance or the endotracheal tube placement. With this suction chamber, the patient will not be in danger of choking on any foreign body or his or her own emesis while breathing assistance is given or while the patient ingests the appropriate medication from the aforementioned dispenser(s).

(2) Discussion of Related Art Including Information Disclosed Under 37 C.F.R. 1.97 and 1.98

The prior art has diverse medical respiratory masks, inhalation devices or nebulizers. However, no device in the prior art comprises all the characteristics or uses of the invention subject of this application.

U.S. Pat. No. 4,119,096 (Drews, 1978) discloses a medical inhalation device for the treatment of diseases of the respiratory tract. This device is a hand held inhalator that involves an electronic connection, unlike the 3-in-1 in-line nebulizer, mediport dispenser and suction chamber for bag valve masks, endotracheal tubes and tracheotomy tubes which is not an inhalator, and which has various functions, such as being a nebulizer dispenser, medication dispenser and suction of foreign bodies or emesis that may be obstructing patient's airway.

U.S. Pat. No. 4,697,587 (Marinkovich, 1987) is for a disposable mouth-to-mouth resuscitation device; U.S. Pat. No. 4,856,548 (Paluch, 1989) is for a resuscitation valve; U.S. Pat. No. 5,349,944 (Chipendale et al, 1994) is for an inhalation device with a reduced risk of blockage; and U.S. Pat. No. 6,328,032 (Virtanen, 2001) claims an inhalation device. All of these prior art inventions are different from the 3-in-1 in-line nebulizer, mediport dispenser and suction chamber for bag valve masks, endotracheal tubes and tracheotomy tubes which is a novel invention that enables a medical professional to provide different treatments (e.g. dispense medication through a medical port, provide nebulizations to patient, and suction foreign bodies that could block the patient's airway). Therefore, these prior art inventions not only are different per se, but have different functions to the 3-in-1 in-line nebulizer, mediport dispenser and suction chamber for bag valve masks, endotracheal tubes and tracheotomy tubes.

U.S. Pat. No. 6,062,217 (Gray, 2000) is for a portable emergency safety resuscitator. This prior art invention discloses a resuscitator, and the invention subject of this application is a 3-in-1 in-line nebulizer, mediport dispenser and suction chamber for bag valve masks, endotracheal tubes and tracheotomy tubes. Therefore, the invention subject of this application has a broader scope than the Gray invention, which is intended to be a resuscitator. Another main difference between these two inventions is that the Gray resuscitator includes a collapsible bag, while the the 3-in-1 in-line nebulizer, mediport dispenser and suction chamber for bag valve masks, endotracheal tubes and tracheotomy tubes is an adjunct that is intended to be compatible for use with any adult or pediatric bag valve masks of different manufacturers and of diverse models in the industry.

### BRIEF SUMMARY OF THE INVENTION

This invention constitutes a 3-in-1 in-line nebulizer, mediport dispenser, suction chamber for bag valve masks and endotracheal and tracheotomy tubes attachment with universal adaptability for airways for adults and children. It enables the user to administer medication orally to the patient, through the nebulizer or aerosolizer. Furthermore, the suction port or chamber prevents the patient from choking on his or her emesis or any other foreign body, and therefore the medical ingestion and breathing or ventilation assistance may continue without interruption and without having to remove the bag valve mask from the patient for dispensing medication to him or her.

### OBJECTS & ADVANTAGES OF THE INVENTION

The present invention constitutes a significant improvement in several aspects over such previously identified efforts of the prior art as described below.

Advantages of the medical inhalation device include and are not limited to providing the medical field, in particular for first aid in ambulances and in hospital emergency rooms, a novel device that will enable a patient, with probable or actual breathing difficulties, to have medication (cardiac or narcotic antagonists) dispensed through an endotracheal tube or tracheotomy tube and through a nebulizer dispenser. Furthermore, the invention has a suction chamber or port that shall facilitate the removal of foreign bodies or emesis from the oral airway or endotracheal tube without jeopardizing ventilation assistance from the endotracheal tube placement.

In view of the foregoing, various objects and advantages of the present invention include the following:

1. One object of the present invention is to provide a device that will enable several medical first aid practices simultaneously, without interruption to patients with probable or current breathing difficulties. It will enable the user to: (i) dispense cardiac medications (such as but not limited to epinephrine, lidocaine, atropine) or narcotic antagonists (such as but not limited to narcan) to a patient through an endotracheal tube or tracheotomy tube; and (ii) to dispense respiratory medications or bronchodilators (such as but not limited to albuterol, brethine and atrovent) to a patient with probable or actual breathing difficulties, through a nebulizer or aerosol dispenser; and (iii) a suction chamber or port to facilitate the removal of emesis from the patient's oral airway or endotracheal tube.

2. This device has the advantage of enabling doctors, nurses and other first aid personnel to dispense medication and aid the patient in breathing without danger of the patient choking on his or her own emesis or other foreign bodies.

3. This device safeguards the oral ventilation or breathing of a patient while medication is being dispensed to him or her.

4. This invention has the advantage that it permits medication to be dispensed to the patient without interruption. The suction chamber or port will remove emesis or oral secretions in the patient's airway, and thus prevent not only the patient from choking but also prevent the interruption of medical dispensing.

5. The suction chamber or port of the 3-in-1 in-line nebulizer, mediport dispenser, suction chamber for bag valve masks, endotracheal and tracheotomy tubes has suction capabilities that enables the removal or emesis, oral secretion or foreign bodies from the patient's airway, and therefore reduces the potential of compromising tube patency, tube displacements or oxygen depravation in patients.

6. This device can be used in emergency situations for adults or in pediatrics.

Other objects, features and advantages will become apparent from a consideration of the detailed description of the invention, accompanying drawings and the claims of the invention.

### BRIEF DESCRIPTION OF THE SEVERAL VIEWS OF THE DRAWINGS

The present application includes 10 additional informal drawings to aid the Examiner in the review of the same. The scope of the 3-in-1 in-line nebulizer, mediport dispenser, suction chamber for bag valve masks, endotracheal and tracheotomy tubes is however limited only by the scope of the claims not by a particular embodiment shown in the drawings.

FIG. 1 is a side view of the 3-in-1 in-line nebulizer, mediport dispenser, suction chamber for bag valve masks, endotracheal and tracheotomy tubes. Here, it is displayed for use in conjunction with a standard bag valve mask (1).

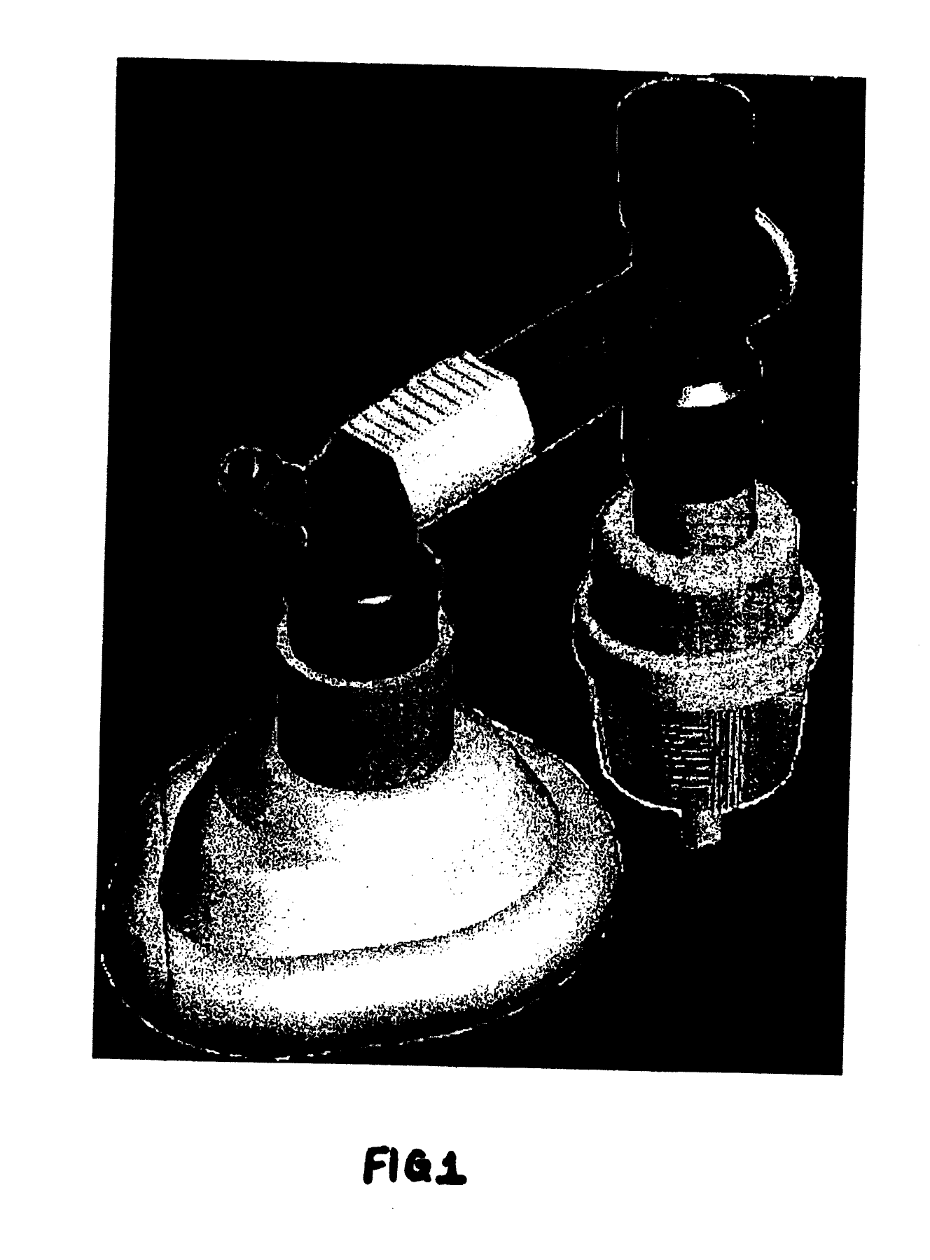

FIG. 2 is a front view of three elements of the novel 3-in-1 in-line nebulizer, mediport dispenser, suction chamber for bag valve masks, endotracheal and tracheotomy tubes: a medication administration port (3), an elbow (5) for bag valve masks, endotracheal tubes and tracheotomy tubes, and a nebulizer chamber (8).

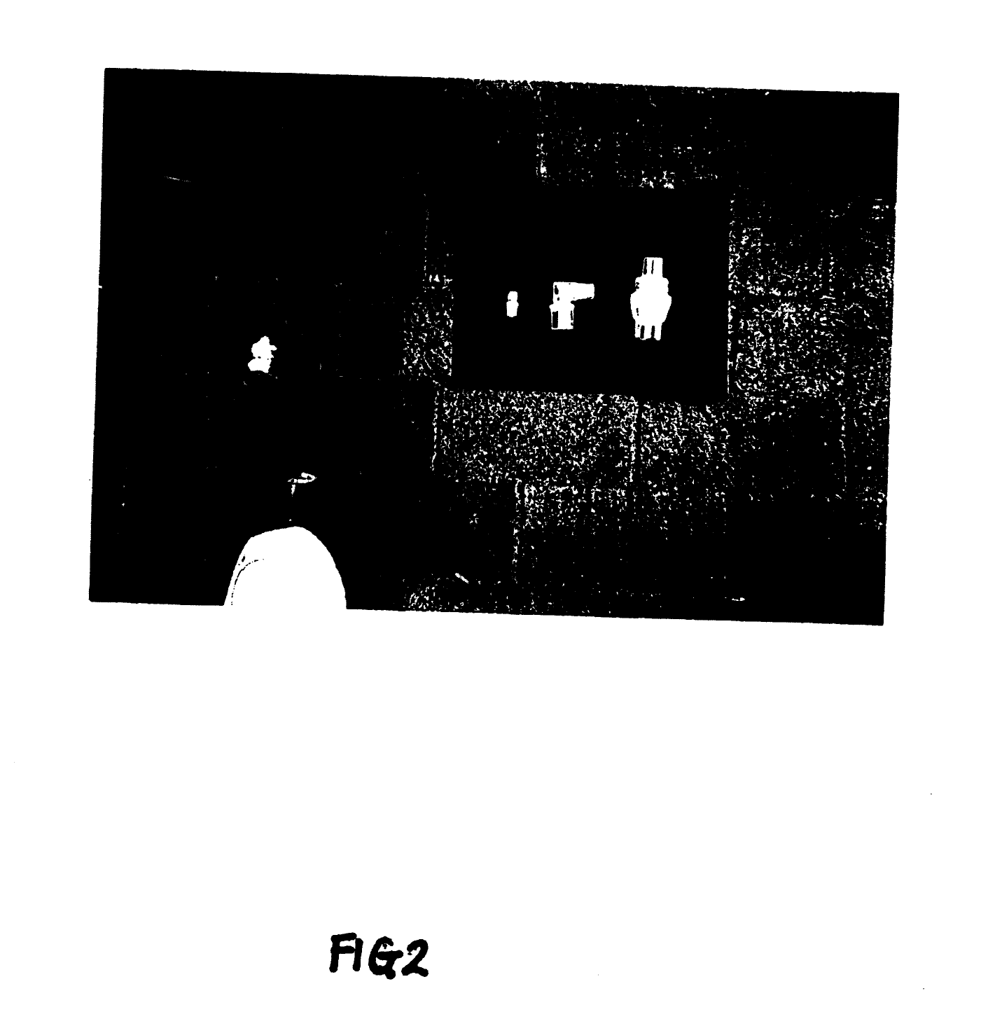

FIG. 3 is a sectional view of the inner sections of the 3-in-1 in-line nebulizer, mediport dispenser, suction chamber for bag valve masks, endotracheal tubes and tracheotomy tubes, and in this embodiment it is being used with a standard bag valve mask (1).

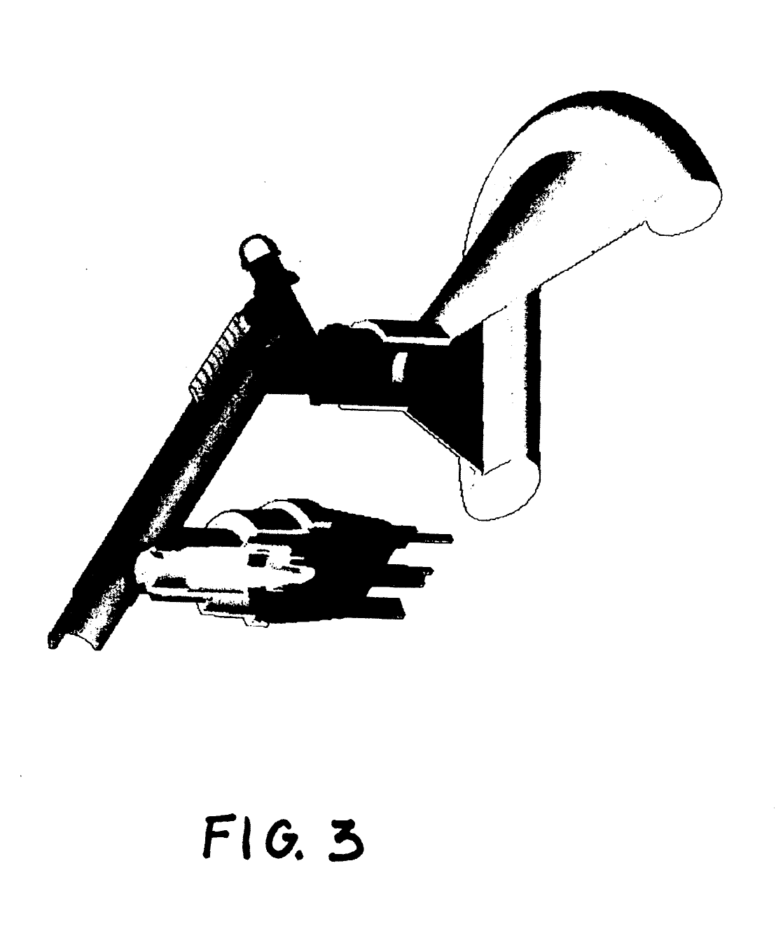

FIG. 4 is a side sectional view of the invention, illustrating the medication administration port (3), the in line nebulizer chamber (8), the in line nebulizer (9) attached to the elbow (5) and the attachment or adjunct for bag valve masks, endotracheal tubes and tracheotomy tubes (2).

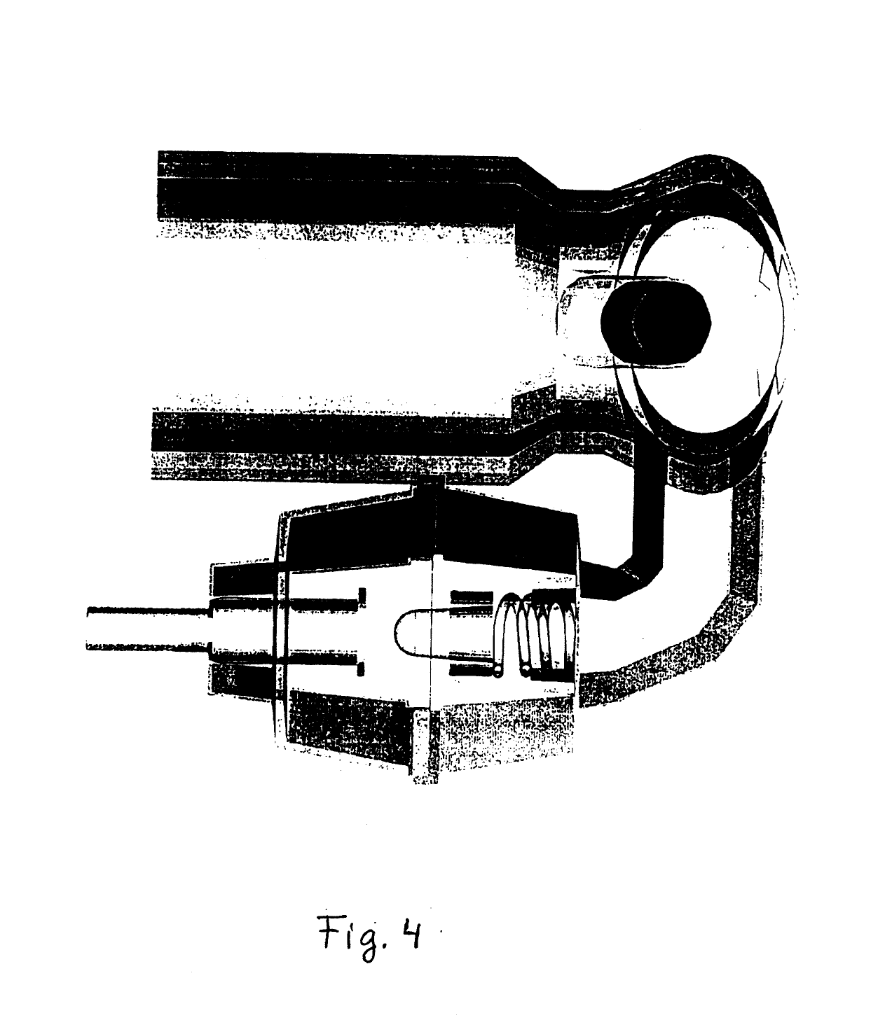

FIG. 5 is a right sectional view of the invention.

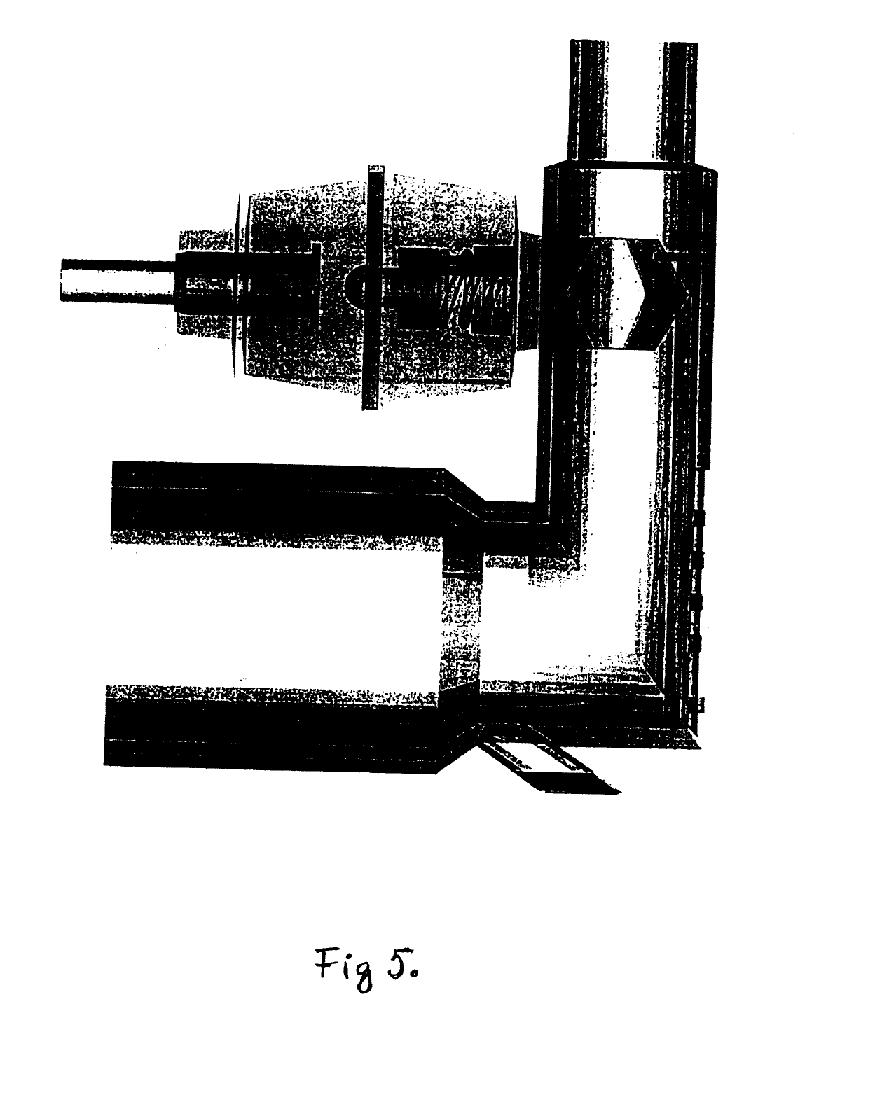

FIG. 6 is a left sectional view of the invention.

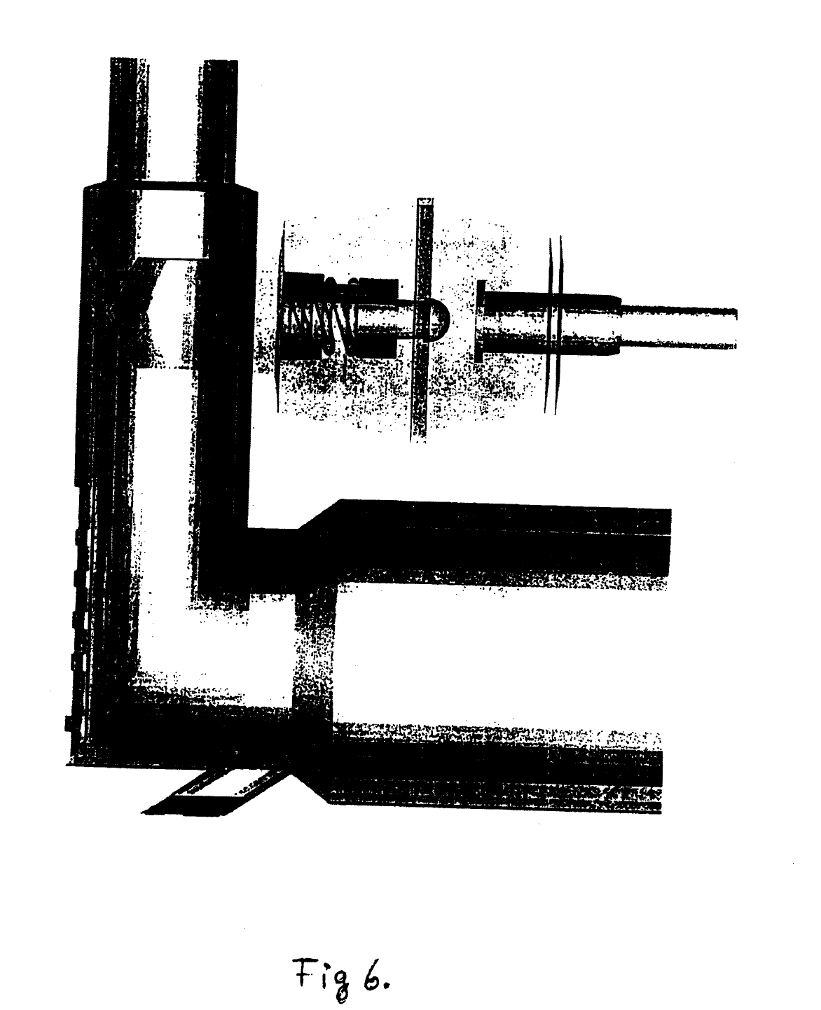

FIG. 7 is a top view of the attachment or adjunct for bag valve masks (6), of the medication administration port (3), of the suction chamber or port (4), of the nebulizer chamber (8), and of the nebulizer (9).

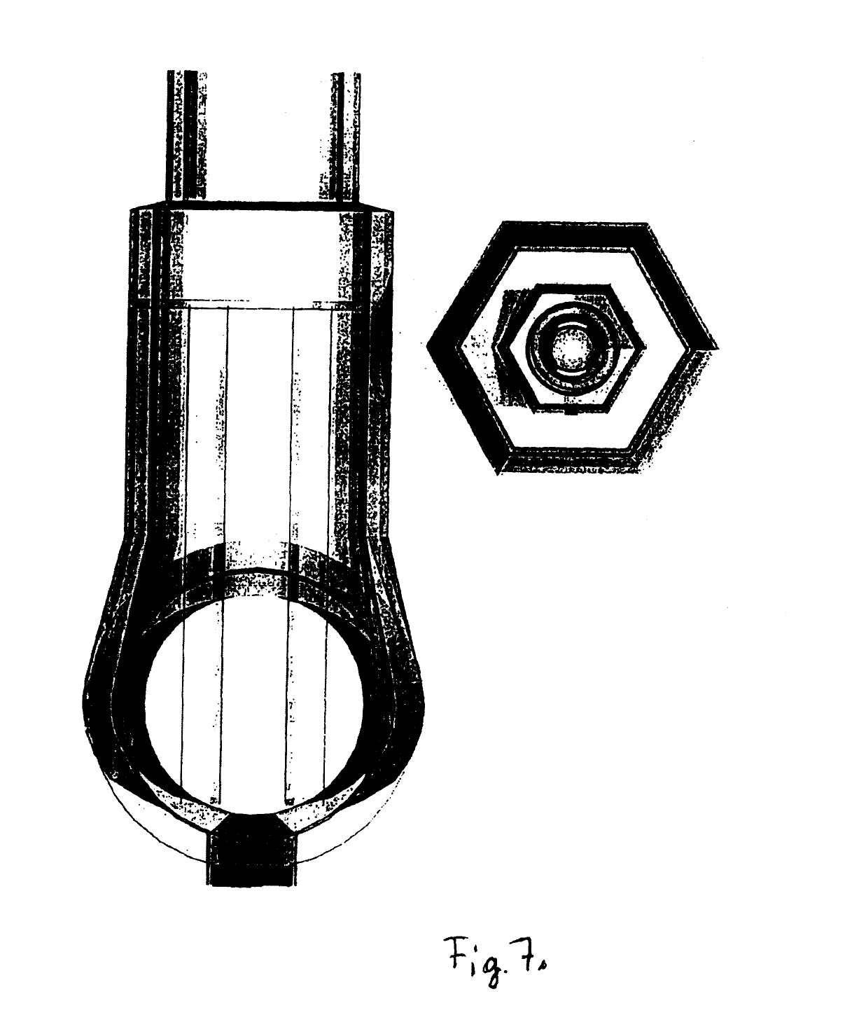

### DETAILED DESCRIPTION OF THE INVENTION

The invention subject of this application comprises a 3-in-1 in-line nebulizer, mediport dispenser, and suction chamber for bag valve masks, endotracheal tubes and tracheotomy tubes attachment with universal adaptability for airways of adults and children. The structural features of preferred embodiment of the device include:

(a) a hollow elbow cylinder (5) with three apertures or connectors; one aperture allowing the elbow (5) to be connected to a retractable suction chamber or port (4) a second aperture (6) allowing the elbow (5) to be sealed with a sealing cap (7) or if open, to connect the elbow (5) to a bag valve mask (1), and a third aperture (11) allowing the elbow (5) to be connected to a nebulizer chamber (8). The elbow (5) is made of plastic and transparent.

(b) The nebulizer chamber (8) comprises an in line nebulizer (9) made of plastic and that may be used for administering in-line bronchodilator medications. In the preferred embodiment of the invention, this element is transparent. Medications and/or aerosol mist are to be facilitated to the lungs via a manually assisted bag valve mask (1) present in the prior art and that can be attached either to the attachment (2) for bag valve masks, endotracheal tubes and tracheotomy tubes or to the adjunct (6) for bag valve masks. The location of the nebulizer chamber (8) laterally branches off the in-line cylinder.

(c) A medication administration port or dispenser (3) that shall be placed in the adjunct or attachment (2) for bag valve masks, endotracheal tubes or tracheotomy tubes. The medication administration port (3) is to be placed in the aperture (10) of the attachment (2) for bag valve masks, endotracheal tubes and tracheotomy tubes. This medication administration port (3) shall expedite the delivery of medications, such as but not limited to cardiac and narcotic antagonist medications down the endotracheal tube or tracheotomy tube of a patient without any need of interrupting ventilation assistance. In this manner, the invention reduces the probabilities of the tube from being dislodged, of tube patency or of oxygen deprivation to the patient on whom the invention is being used. The preferred embodiment is made of plastic and transparent.

(d) A retractable suction port or chamber (4) connects the elbow (5) to the adjunct or attachment (2) for bag valve masks, endotracheal tubes or tracheotomy tubes. It facilitates the removal of foreign bodies or emesis from the oral airway, endotracheal tube or tracheotomy tube without jeopardizing ventilation, breathing assistance or the endotracheal or tracheotomy tube placement. Suction shall be performed via a retractable mechanism (4) located at the top of the elbow (5) and directly above the oral cavity or endotracheal tube or tracheotomy tube.

### OPERATION OF INVENTION

The invention is operated by placing the hollow elbow cylinder (5) with its attached nebulizer chamber (8), suction chamber or port (4), and adjunct (2) for bag valve masks, entracheal tubes or tracheotomy tubes into the openings of bag valve masks (1), endotracheal tubes or tracheotomy tubes of patients. The invention may be used for administering in-line bronchodilator medications through its nebulizer chamber (8) and inline nebulizer (9). Medications or aerosol mist shall be facilitated into the lungs via a manually assisted bag valve mask (1).

The medical administration port (3) may be placed at the aperture (10) of the attachment (2) for bag valve masks, endotracheal tubes and tracheotomy tubes, and it will allow cardiac and narcotic antagonist medications to be dispensed to the patient, down the endotracheal tube or tracheotomy tube without any need of interrupting ventilation or breathing assistance to the patient. Therefore, the invention reduces the potential of the endotracheal tube or tracheotomy tube from being dislodged, from tube patency or from depriving of oxygen to the patient.

The retractable suction port or chamber (4) shall facilitate the removal of foreign bodies or emesis from the oral airway, endotracheal tube or tracheotomy tube without jeopardizing ventilation, breathing assistance or the endotracheal or tracheotomy tube placement. Suction shall be performed via a retractable mechanism or suction port (4) located at the top of the elbow (5) and directly above the oral cavity, endotracheal tube or tracheotomy tube. In the preferred embodiment of the invention this element is made of plastic and is transparent.

### DESCRIPTION AND OPERATION OF ALTERNATIVE EMBODIMENTS

The invention subject of this application comprises a 3-in-1 in-line nebulizer, mediport dispenser, and suction chamber for bag valve masks, endotracheal tubes and tracheotomy tubes attachment with universal adaptability for airways for adults and children. The invention may be made of different materials and sizes (for adults and children), therefore it may have different embodiments. The main features of the device include:

(a) A hollow elbow cylinder (5) with three apertures or connectors; one aperture allowing the elbow (5) to be connected to a suction chamber or port (4), a second aperture (6) allowing the elbow (5) to be sealed with devices such as but not limited to a cap (7) or if open to connect the elbow (5) to a bag valve mask (1), and a third aperture (11) allowing the elbow (5) to be connected to a nebulizer chamber (8). The elbow (5) may be made of materials such as but not limited plastic and/or any man made materials. In addition, it may be transparent or of any color.

(b) The nebulizer chamber (8) comprises an in line nebulizer (9) or an aerosol dispenser that may be used for administering in-line bronchodilator medications. This cylinder (9) may be made of a materials such as but not limited to plastic and/or any other man made material. It may be transparent or of any color. Medications and/or aerosol mist are to be facilitated to the lungs via a manually assisted bag valve mask (1) of the prior art and that can be attached to either of the adjuncts for bag valve masks (2) or (6). The location of the nebulizing chamber is laterally branching off the in-line cylinder.

(c) A medication administration port or dispenser (3) placed to the adjunct or attachment (2) for bag valve masks, endotracheal tubes and tracheotomy tubes. The medication administration port (3) shall expedite the delivery of medications, such as but not limited to cardiac and narcotic antagonists down the endotracheal tube or tracheotomy tube without need of interrupting ventilation assistance. In this manner, the invention reduces the probabilities of the endotracheal tube or tracheotomy tube from being dislodged, from tube patency or of oxygen deprivation to the patient on whom the invention is being used. The medication administration port (3) may be made of materials such as but not limited to plastic or any man made material and may be transparent or of any color.

(d) A suction port or chamber (4) that shall facilitate the removal of foreign bodies or emesis from the oral airway, endotracheal tube or tracheotomy tube without jeopardizing ventilation, breathing assistance and tube placement. This shall be performed via a retractable mechanism (4) located at the top of the elbow (5) and directly above the oral cavity, endotracheal tube or tracheotomy tube. This suction port (4) may be made of materials such as but not limited to plastic or any man made material and may be transparent or of any color.

The invention is not however limited to the above embodiments and materials, for they are given as examples only. The scope of the invention should be determined by its claims not by a particular embodiment of the invention.

### CONCLUSION, RAMIFICATION & SCOPE OF INVENTION

This device subject of this application offers a unique 3-in-1 in-line nebulizer, mediport dispenser, and suction chamber for bag valve masks, endotracheal tubes and tracheotomy tubes attachment with universal adaptability for airways for adults and children. A suction port (4) facilitates the removal of foreign bodies or emesis from the oral airway or endotracheal tube of the patient without jeopardizing ventilation or breathing assistance. The scope of the invention described here is for example only. The scope of the invention shall be determined as described within the claims of the invention.

I claim:

1. A respiratory and medicine dispensing attachment to be used in conjunction with bag valve masks, endotracheal tubes and tracheotomy tubes present in the prior art, comprised of:
a hollow elbow cylinder with three apertures or connectors; one aperture allowing the elbow to be connected to a retractable suction chamber or port, wherein this suction chamber can remove foreign bodies, oral secretions or emesis from the patient's airway or from any tube; a second aperture that may be sealed by user with a cap and if open, allow the elbow to be connected a bag valve mask for aiding respiration or administration of medication, and a third aperture allowing the elbow to be connected to a nebulizer chamber which includes an in line nebulizer or aerosol dispenser for administering medications;
an attachment or adjunct for the elbow, having this adjunct two apertures, one for the attachment of bag valve masks, endotracheal tubes and tracheotomy tubes, and the second one for the placement of a medication administration port or dispenser;
a medication administration port or dispenser attached to the adjunct or attachment for bag valve masks, endotracheal tubes or tracheotomy tubes, wherein the medication administration port is placed in the second aperture of the attachment for bag valve masks, endotracheal tubes and tracheotomy tubes; the retractable suction port or chamber connects the elbow to the adjunct or attachment for bag valve masks, endotracheal tubes and tracheotomy tubes, wherein this suction port facilitates the removal of foreign bodies, oral secretions and emesis from the patient's oral airway, or the placed endotracheal tube or tracheotomy tube without jeopardizing ventilation, breathing assistance or the endotracheal or tracheotomy tube placement, and wherein suction is performed via a retractable mechanism located at the top of the elbow and directly above the oral cavity or endotracheal tube or tracheotomy tube being used.

2. The invention described in claim 1 wherein the invention is made of materials such as but not limited to plastic and/or man made materials.

3. The invention claimed in claim 1 wherein the invention is of any size, therefore, being able to be used by adults or children.

4. The invention described in claim 1 wherein the second aperture of the hollow elbow cylinder is sealed with a device such as but not limited to a cap.

5. The invention described in claim 1 wherein the elements are transparent or of any color.

6. The invention described in claim 1 wherein the nebulizer chamber and in line nebulizer or aerosol dispenser may be used for administering medications such as but not limited to bronchodilators.

7. The invention described in claim 1 wherein the medications and/or aerosol mist may be facilitated to the patient's lungs via a manually assisted bag valve mask present in the prior art and that may be attached to either attachments of the invention, being these attachments the adjunct for bag valve mask, Endotracheal tubes and tracheotomy tubes or the attachment for bag valve masks.

8. The invention described in claim 1 wherein the medication administration port expedites the delivery of medications, such as but not limited to cardiac and narcotic antagonists down the endotracheal tube or tracheotomy tube of a patient without any need of interruption ventilation assistance, reducing the probabilities of the endotracheal tube or tracheotomy tube from being dislodged, from tube patency or of oxygen deprivation to the patient on whom the invention is being used.

9. The invention described in claim 1 wherein the retractable suction port or chamber facilitates the removal of foreign bodies, oral secretions or emesesis from the oral airway, endotracheal tube or tracheotomy tube without jeopardizing ventilation, breathing assistance or the endotracheal or tracheotomy tube placement.
